SEQUENCE LISTING (17 sequences)

SEQ ID NO: 1 - DNA, 4632 bp, Helicobacter bilis
  P167
atgagagtgaaagcatatatattaagtgggattttgcttagtgtgagtcattgtgtatatgctggggatattcaaggcgatatttataatctaaatagcaaaaatgatagctcaagcgatacattttatagtaacaatatggggacaacatacactggtttggcatggggtagagattttacacaaagttatgaaaatggtacattaatcattggcaacacttcaaaaagtccaacaagtaacggacattggtttggtcgtggcggtgatgtagggtttattaatggtactttcaacgcaaaagaagtctatattacaggtacattaggttctggtaatgcagcaaggacaggtggcggggcaaatcttacttttaatgcaagcaataatctcacattagatggggcaaatatcagtgtaaatcgtgcaggcacacaaaattcaactactgctttaaatggcaaagaaatcgatataaaaaattcacaattcaatatagaaaatatcaatggtggtggcattaatatcggcaatgaaaaaacagagaatctaactatagataatacaaatataagtatgagtggcggacagataaatatcaccgcaaaaaactcaaagctaaactttgggtcggtcaatatcacaaatggcacacttgatttaacaaaagcaaattatacagaacttacaacacactcacttaatatcacaaactctagctttcgctcaaatgagctaaatatgtcaggtgatattgtaaatggtagattcacaccaacacaaactctagcacatggggcaggttttcgccaaagtctaggaaatggaggggatttcactgcggattctataacaatagatgatggcagtagcaccttaaatgcgactaatgcgacgattaaagagttaaatttcaaaggtccatcaacaatctttggttcaacacaaaaaaagcttactgctactattagcggtaagttaaatattacaacactcacaaatccttaccaaccgtttggtaaaggttttgccgctgaactagaagtaaaacaggctagtgaagtcaatatagacaatatacaccaccaaaactatacgggcggtagctggatacatgtcaatgtggataacaaacttgttgtaggaaatattcagtttgcaacagatggtatattgggaagtcaagggcatgtcgagcttaaatctagtaacggcgatgtaatagtaaaaaatgcagctggttgtggcaatgcctgcacaggtggtttaggtattcgtccgctaaattttcttaaaatatctggtaaaaatttctatggtggcaacatagaagctataggtttgccaacagctggaagcaattctacgcttgatttaacaggggttagcggcacaaaatttatcgatactttctctatacgcacaggcacgctacaagcaaaagattttcatgtgaataattttattgtctataaaagtaactcttactctgtagtagatcaaaatatcggtaaaagtttcatcaactatctctcaatggaaataggcacaagcgactttgcagatggagcagatattagattcaatggtgggggtgagattctaaactttaacttcatcgatgcgagaccgacttcatttgtgcgtatgggtgagattaagcatgtcaatgttaatgaaatgaaaatcaaagaagctgaagtcaatattaaaaatggaatctttaatacaatagtttcacaaaaagggcaaaccgctatcacaaacggcgcttcaaaaatcacagctaacgccctaaatgtcaatgagcttttacacctaaaagactcgagcaagcatagcggtgttatgaaaattgagttaaatgctaatggcgaaaacatagaccaaaagtttgacaaatacctaaatgtaagcgtagattctaaagacctaaaaaatatgagtggtgatgagatagcaaagcttatagaatctaataaaaacacaaatggaaataatgaaaaaacagaatcagattacaaaaacgcaagtggcacatatatcacaacaagtgatggcaaacattatctagtcttgccagatattatgacaaatgataatattacaaatagcacaggacaagttgcaaatggtggtaatatcttaatagagcaaaccgaactcacaaaaggtgcattaaataactatggaaagattctaatcgatgatggctttgtaagtggagataaaccgacactctatctcacaggtaatctcaataactacaatacaatagatttgggagctaacggacatatagaagtaacaggaaactttaataacaaaggtaaaatcctttttagcattcaagcaaatccaaatgactctagcaacaatgaaataaaaaatgcaagtatgaatatcaaaggcaaagcaacactagacatatcacccggaagcacaggggcattacaggcagatatagcagattctaagactttatcgagtataagcagtcagcttaatactaatggtagcaagggcgtagaatacaatctcataaaagcagaatctataagctacacttacacacaaggggattatactacgacttttgataaaagcaacggcaatacaaccataaaaacagaacacaccaatggcggacaatgcacaaataaagactgccaaacaggaagcaatggcaatacaaaacaagaaaccgacttcacaacaaatggcaaagacaatccatgggatatggataaagcccaaacagattctaatggcaatgtagtagaaaatggcataggctcaactgataatgaaggcaatctcatacaaaagccaagtcagcaagataaacaagaagaatgcaaacaagatggcaagtattgtggctttggtagcaatgctaacgctactgaagcaaacagaggctatgattacgcacaacaaaagatgaaaaatagcttttcactcacttataaaggcggtagcatagatggtaaatatatcgatatagaaaaagtcgtaaatgatacaacaataggctttaaaatcaataaagtagatattaataacttcatagagggcaatacagaaaagcctttatgtaatgcagaatctagcacatttgattgtgctttatacatggaagcaggtggaaataactcatggataaatgcgattaaaaaagagagtgcaaatagctatgagattctaaaaaatctattctatgatgataaatcttcacttgtgtttttaataaacctagaccaaactctagctgcctcaagaaacctagagtatttcctagaagtaggtaggactctagatacagcaatagatcatgtctcaaacctagagaataaagcatcaacacttcatacactcacactatcaatggagagtgcaaagctaaatcgtctagcaagagtagctagcattcatggtagtgctagtagctatatagctatgcaaaactatcaaaagaatttagaagcagcattaagggaagcagagaagctaaaactagcaagtctagattctaatacaaacacaagaatagcagctattaataatatagtaagtggtaatacaaatagtaatgcagtttatagaagtagtgtagctactctagcaagtagctctaacacaaacacacaaaatgcaaacaatggagtaacagatatacaaagtgatgttacaactgataacaactcaaatgaagtatggtttgataccttagcagatttaatcatgagatttaataatagagaagaatatccaaatcatgcttgggttaatatgctaggtaatcttaacttctcaaagactggctcggctcaactttatggctttaatgcaggatatgattattttgtagataaactccaaactgcatttggtgcgtatggtggttatggctatggcacatttaatggcaataataatggctttacttcaaataattcaaacaatatgtttgctggaatctatacaaggactttcattgaaaaccatgaaatagatgtaacactcaatactgcctttagctttacaaatgagaaacaaaactctcaaattaataactttgacttacttgctctctttaaagatgagtatagctacacactctctaatgtagatttaaatgcaacttatggttatgcttttttaatgaaaaagggttatattgtaaaaccttttgcaggattatcgtattatgtattaggctcaagtggctttaaaagaaagagtgataatacacctatatttgctatgaatacagatgataacataagacaaactataagcgtaaatataggtatagatggtcgtaaatactttgcaaatcaaagctatatttttatagtcgctcaattaaagcaagatgcaatcattttacaaaataatgtagattctagtgggactacaactacaagtataggcaatataggtgattctgtaaataactttaatatgagatataaagctcaaggctataagtcttatgtattccttacaggtggtggagaatatagctttggtagatggtatttaaatggcagtcttagcttacaaagcagtgtatttgataaaaactttggcttaggatttaatattggtggtagggttgtgttttag

SEQ ID NO: 2 - DNA, 24 bp, Artificial Sequence
  Description of Artificial SequenceHelicobacter bilis P167A 5′ primer
tatgctggggatattcaaggcgat

SEQ ID NO: 3 - DNA, 21 bp, Artificial Sequence
  Description of Artificial SequenceHelicobacter bilis P167A 3′ primer
attgacatgtatccagctacc

SEQ ID NO: 4 - DNA, 21 bp, Artificial Sequence
  Description of Artificial SequenceHelicobacter bilis P167B 5′ primer
atagatgatggcagtagcacc

SEQ ID NO: 5 - DNA, 21 bp, Artificial Sequence
  Description of Artificial SequenceHelicobacter bilis P167B 3′ primer
tgctatctcatcaccactcat

SEQ ID NO: 6 - DNA, 24 bp, Artificial Sequence
  Description of Artificial SequenceHelicobacter bilis P167C 5′ primer
cgtatgggtgagattaagcatgtc

SEQ ID NO: 7 - DNA, 24 bp, Artificial Sequence
  Description of Artificial SequenceHelicobacter bilis P167C 3′ primer
tgagcctatgccattttctactac

SEQ ID NO: 8 - DNA, 24 bp, Artificial Sequence
  Description of Artificial SequenceHelicobacter bilis P167D 5′ primer
agctacacttacacacaaggggat

SEQ ID NO: 9 - DNA, 21 bp, Artificial Sequence
  Description of Artificial SequenceHelicobacter bilis P167D 3′ primer
atctgttactccattgtttgc

SEQ ID NO: 10 - DNA, 24 bp, Artificial Sequence
  Description of Artificial SequenceHelicobacter bilis P167E 5′ primer
cgtctagcaagagtagctagcatt

SEQ ID NO: 11 - DNA, 24 bp, Artificial Sequence
  Description of Artificial SequenceHelicobacter bilis P167E 3′ primer
attggtggtagggttgtgttttag

SEQ ID NO: 12 - DNA, 705 bp, Helicobacter hepaticus
  P25Hh4
atgggtaagaaaatagcaaaaagattgcaaactttacaggcaaaagttgagcctcaaaaggtttatagcattcaaagtggtgtgagcgcagtgaagtctttggcttctgcaaaatttgatgaaacggtagaagtagcattacgtttaggagtagatcctagacacgctgaccaaatgatacgtggagcagtagttttgccacacggaacaggtaaaaaagtgcgtgttgctgtgtttgcaaagggtattaaggctgatgaagcaaaaaatgcaggagctgatgtagtgggtgctgatgatttggcagaagagattaaaaatggcaatattaactttgatatggttattgctactcctgatatgatggctcttgtaggtaaagtggggcgtattttgggaccaaagggtttaatgccaaatccaaaaacaggcacagttacaatagatgttgctaaagctgtagctaacgcaaaaagcggacaagtaaattttagagttgataaaaagggtattatccacgcacctattggtaaagcttcttttaatgaagagaagattttagataatatgcttgaacttgtgcgtgctatcaataggcttaagcctacttccgcaaaaggtaaatatattcgcagcagtagtttatcgctcacaatgagtccagcaattaagcttgattctcaagagcttatggatatgaaatag

SEQ ID NO: 13 - DNA, 30 bp, Artificial Sequence
  Description of Artificial SequenceHelicobacter hepaticus P25Hh4 5′ primer
atgggtaagaaaatagcaaaaagattgcaa

SEQ ID NO: 14 - DNA, 30 bp, Artificial Sequence
  Description of Artificial SequenceHelicobacter hepaticus P25Hh4 3′ primer
ctatttcatatccataagctcttgagaatc

SEQ ID NO: 15 - DNA, 435 bp, Helicobacter bilis
  P17Hb48
atggaacagataaagattttaaagcaacttcaggcagattcattagttttctttacaaaaactcacaattaccactggaatgtaaaaggcaaagattttccacaagtccatgcggcaacagaagagatttataatcaattcgcagaaatctttgatgctctagcagagagaatcatacagcttggcgacactccgtatgtaacgctaaaagaagtgcttgataaagcaaagattaaagaagaaagcaaaactagttttaagtcaaaagatgtattagaatccgttttagaagattacaaatatttcctaaaaaactttaaaaaactttcagaagtggcagcaaaagagaatgncactncaacacaaggatttgcagattcacaagtagcacatttagaaaaagcaatttggatgcttaacgcacaacttgcatag

SEQ ID NO: 16 - DNA, 33 bp, Artificial Sequence
  Description of Artificial SequenceHelicobacter bilis P17Hb48 5′ primer
atggaacagataaagattttaaagcaacttcag

SEQ ID NO: 17 - DNA, 24 bp, Artificial Sequence
  Description of Artificial SequenceHelicobacter bilis P17Hb48 3′ primer
ctatgcaagttgtgcgttaagcat

(19) United States
(12) Patent Application Publication
(10) Pub. No.: US 2003/0224401 A1
(43) Pub. Date: Dec. 4, 2003

(54) Proteins for helicobacter diagnosis

(75) Inventors: Stephen Barthold, Davis, CA (US); Sunlian Feng, Davis, CA (US); Emir Hodzic, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, Oakland, CA

(21) Appl. No.: 10/370,864

(22) Filed: Feb. 19, 2003

Related U.S. Application Data
(60) Provisional application No. 60/358,333, filed on Feb. 19, 2002.

(51) Int. Cl. C12Q 1/68; G01N 33/554; G01N 33/569; C07H 21/04; C12N 9/78; C12P 21/02; C12N 1/21

(52) U.S. Cl. 435/6; 435/7.32; 435/69.3; 435/252.3; 435/320.1; 435/227; 536/23.2; 530/388.26

(57) ABSTRACT

The present invention provides isolated polynucleotides encoding the Helicobacter bilis antigens P167 and P17Hb48 and the Helicobacter hepaticus antigen P25Hh4. The present invention further provides methods and kits for detection and diagnosis of Helicobacter infections using the polynucleotides and polypeptides described herein.

### CROSS-REFERENCES TO RELATED APPLICATIONS

This application claims the benefit of U.S. Provisional Application No. 60/358,333, filed Feb. 19, 2002, the disclosure of which is incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant No. RR14034, awarded by the PHS/NIH. The Government has certain rights in this invention.

### REFERENCE TO A “SEQUENCE LISTING,” A TABLE, OR A COMPUTER PROGRAM LISTING APPENDIX SUBMITTED ON A COMPACT DISK

Not applicable.

### FIELD OF THE INVENTION

This invention relates to Helicobacter proteins for diagnosis of Helicobacter (e.g., H. bilis and H. hepaticus) infections.

### BACKGROUND OF THE INVENTION

Helicobacter bilis is a member of an expanding and genetically diverse group of enterohepatic, commensal and opportunistic Helicobacters that infect laboratory mice. In addition to H. bilis, Helicobacters isolated from mice include H. hepaticus (Fox, et al. (1994) J. Clin. Microb. 32: 1238-1245), H. rodentium (Shen, et al. (1997) Int. J. Syst. Bacteriol. 47: 627-634.), H. muridarum (Lee, et al. (1992) Int. J. Syst. Bacteriol. 42: 27-3), H. typhlonicus (Franklin, et al. (1999) Lab. Anim. Sci. 49: 496-50), and others yet to be named (Shomer, et al. (2001) Exp Biol Med. 226: 420-428). Flexispira rappini, first described by Bryner, et. al (Bryner, et al. (1986) XIV International Congress of Microbiology, Manchester, UK), has also been shown to be a Helicobacter (Vandamme, et al. (1991) Int. J. Syst. Bacteriol. 41: 88-103) (Paster, et al. (1991) Int. J. Syst. Bacteriol. 41: 31-38) (Schauer, et al. (1993) J. Clin. Microb. 31: 2709-2714), but recent studies suggest that it represents a mixture of Helicobacter species (Dewhirst, et al. (2000) Microbio. 50: 1781-1787). H. bilis infection has been found to be widespread among research mouse colonies (Fox, et al. (1995) J. Clin. Microb. 33: 445-454) (Riley, et al. (1996) J. Clin. Microb. 34: 942-946). Infections are often subclinical, but can produce liver and enteric disease in some genotypes of mice, and particularly in mice with immune deficiencies. H. bilis was initially isolated from aged inbred mice with chronic hepatitis and hepatomas in 1995 (Fox, et al. (1995) J. Clin. Microb. 33: 445-454), then subsequently isolated from SCID mice with enteritis that were co-infected with H. rodentium (Shomer, et al. (1998) Lab. Anim. Sci. 48: 455-459). Experimental inoculation of H. bilis induces enteritis and hepatitis in SCID mice (Shomer, et al. (1997) Infect. Immun. 65: 4858-4864) (Franklin, et al. (1998) Lab. Anim. Sci. 48: 334-339), and enteric disease in athymic rats (Haines, et al. (1998) Vet. Pathol. 35: 202-208). The enteric lesions that are now known to be associated with Helicobacter infection in mice, including H. bilis, are useful as models of human inflammatory bowel disease (Strober and Ehrhardts. (1993) Cell. 75: 203-205). Genomic alteration of mice can have both intentional and unpredicted immune perturbations that enhance the pathogenicity of these opportunistic pathogens. In addition to infecting mice, H. bilis has been isolated from dogs, gerbils, rats, and cats (Ge, et al. (2001) Inf. Immun. 69: 3502-3506), and its DNA has been amplified from bile and gall bladders of humans with cholecystitis (Fox, et al. (1998) Gastroenterology. 114: 755-763).

Sera from mice infected with H. bilis react with a number of proteins that could potentially serve as antigens for serodiagnosis, if cloned and expressed in recombinant form. Membrane antigens are logically favored targets, in that it is presumed that they are most apt to interface with the host during infection and thus elicit an antibody response. In one study, nine native proteins from H. bilis outer membrane protein (OMP) preparations were found to be immunoreactive. The proteins ranged from 20 to 80 kDa (Fox, et al. (1998) Gastroenterology. 114: 755-763).

For these reasons, there is need for diagnostic assays that are both specific and sensitive. Currently available serologic assays for detecting Helicobacter infection in mouse populations have relied on either bacterial lysates (Fox, et al. (1996) Infect. Immun. 64: 1548-1558; Fox, et al. (1996) Lab. Anim. Sci. 64: 3673-3681; Ward, et al. (1994) Am. J. Pathol. 145: 959-968) or various types of membrane antigen preparations (Livingston, et al. 1997. J. Clin. Microb. 35: 1236-1238; Whary, et al. (2000) Comp. Med. 50: 436-443; Ge, et al. (2001) Inf. Immun. 69: 3502-3506). Both are antigenically complex, with cross-reactive antigens causing lack of specificity (Whary, et al. (2000) Comp. Med. 50: 436-443). In addition, these antigen preparations generally detect only low titers of serum reactivity in naturally infected mice, and are not useful for detecting early stages of infection (Fox, et al. (1996) Infect. Immun. 64: 1548-1558; Fox, et al. (1996) Lab. Anim. Sci. 64: 3673-3681; Livingston, et al 1997. J. Clin. Microb. 35: 1236-1238). Both fecal culture and PCR have been shown to detect infection several weeks before positive membrane antigen seroconversion in sequentially sampled, experimentally H. bilis-infected mice (Hodzic, et al. (2001) Comp. Med. 51: 406-412).

Membrane extracts are currently being used for ELISA antigens for Helicobacter serodiagnostic purposes, but results have not been particularly sensitive or specific. For example, serum from mice experimentally infected with H. hepaticus cross reacted with H. bilis and H. rodentium membrane extracts, making the specificity only 34%, and 35%, respectively (Whary, et al. (2000) Comp. Med. 50: 436-443). Sera from mice that were naturally exposed to H. bilis had cross reactivity with H. hepaticus membrane antigen by ELISA, and PCR and culture had to be performed to determine if mice were coinfected (Whary, et al. (2000) Comp. Med. 50: 436-443).

Recently, a H. hepaticus recombinant immunogenic protein (MAP18) was cloned and expressed (Livingston, et al. (1999) Clin. Diagn. Lab. Immunol. 6: 745-750). It proved to be H. hepaticus-specific, but less sensitive than membrane extract antigen (Livingston, et al. (1999) Clin. Diagn. Lab. Immunol. 6: 745-750). No recombinant proteins of H. bilis have been characterized. There is a need in the art to identify novel recombinant H. bilis and H. hepaticus gene products with potential for use as a specific and sensitive diagnostic antigens. The present invention addresses these and other needs.

### BRIEF SUMMARY OF THE INVENTION

This invention provides polynucleotides, polypeptides, methods, and kits useful for detecting Helicobacter infection (e.g., Helicobacter bilis and Helicobacter hepaticus). The polynucleotides, polypeptides, methods, and kits of the invention provide more sensitive and specific means for detecting and diagnosing H. bilis and H. Hepaticus infections.

One embodiment of the invention is an isolated polynucleotide that hybridizes under stringent conditions to the sequence provided in SEQ ID NO:1, nucleotides 25-1122 of SEQ ID NO:1, nucleotides 856-1962 of SEQ ID NO:1, nucleotides 1645-2799 of SEQ ID NO:1, nucleotides 2560-3675 of SEQ ID NO:1, nucleotides 3409-4632 of SEQ ID NO:1, SEQ ID NO:12, SEQ ID NO:15, or a complement thereof. Expression vectors comprising the polynucleotide and host cells comprising the expression vector are also provided. Isolated polypeptides encoded by SEQ ID NO:1, nucleotides 25-1122 of SEQ ID NO:1, nucleotides 856-1962 of SEQ ID NO:1, nucleotides 1645-2799 of SEQ ID NO:1, nucleotides 2560-3675 of SEQ ID NO:1, nucleotides 3409-4632 of SEQ ID NO:1, SEQ ID NO:12, SEQ ID NO:15, or a complement thereof are also provided. Primers specific for particular H. bilis and H. hepaticus proteins or subsequences thereof are also provided.

Another embodiment of the invention is method for detecting Helicobacter infections. In a preferred embodiment, the Helicobacter infection is detected in rodents. A polypeptide encoded by SEQ ID NO: 1, SEQ ID NO:12, SEQ ID NO:15, or an immunogenic fragment of said polypeptide is contacted with a biological sample. A complex is formed between the polypeptide and an antibody in the sample. The presence of the complex is detected, thereby detecting the presence of Helicobacter infection. The method may also be carried our using the polypeptide encoded by nucleotides 1645-2799 of SEQ ID NO 1 or the polypeptide encoded by nucleotides 2560-3675 of SEQ ID NO:1. The method may also include the step of contacting the complex with a rodent Ig-specific antibody. The rodent Ig-specific antibody may be labeled with a detectable label (e.g., an enzyme, a radioisotope, a fluorescent label, or biotin). The rodent Ig-specific antibody may be a mouse Ig specific antibody or a rat Ig-specific antibody. The biological sample may be diluted 1:800, 1:1600, 1:3200, or 1:6400 before the step of contacting the polypeptide and the biological sample.

Yet another embodiment of the invention is a kit for detecting Helicobacter infection. In a preferred embodiment, the kit is useful for detecting Helicobacter infection in rodents. The kit contains a polypeptide encoded by SEQ ID NO:1, SEQ ID NO:12, SEQ ID NO:15 or an immunogenic fragment of such a peptide. The kit may also contain a rodent Ig-specific antibody. The rodent Ig-specific antibody may be labeled with a detectable label (e.g., an enzyme, a radioisotope, a fluorescent label, or biotin).

A further embodiment of the invention is a method for detecting Helicobacter infection. A target nucleic acid sequence is amplified with a first primer and a second primer specific for Helicobacter nucleotide sequence and the amplified product is detected. In one embodiment, the method is useful for detecting Helicobacter infection in rodents. In some embodiments, the target nucleic acid is from H. bilis. In some embodiments, the first primer hybridizes to the sequence set forth in SEQ ID NO:16 and the second primer hybridizes to the sequence set forth in SEQ ID NO:17. In some embodiments, the first primer comprises the sequence set forth in SEQ ID NO:16 and the second primer comprises the sequence set forth in SEQ ID NO:17. In some embodiments, the first primer and the second primer are independently selected from the group consisting of SEQ ID NOS:2, 3, 4, 5, 6, 7, 8, 9, 10, 11, 12, 16, and 17. In some embodiments, the target nucleic acid is from H. hepaticus. In some embodiments, the first primer hybridizes to the sequence set forth in SEQ ID NO:13 and the second primer hybridizes to the sequence set forth in SEQ ID NO:14. In some embodiments, the first primer comprises the sequence set forth in SEQ ID NO:13 and the second primer comprises the sequence set forth in SEQ ID NO:14. The invention further provides an isolated nucleotide comprising the sequence set forth in SEQ ID NOS: 2, 3, 4, 5, 6, 7, 8, 9, 10, 11, 12, 13, 14, 16, or 17.

Other embodiments and advantages of the present invention will be apparent from the detailed description that follows.

### BRIEF DESCRIPTION OF THE DRAWINGS

FIG. 1 illustrates the relative size, inter-relationship, and location of P167A-E peptides in reference to the entire P167 molecule.

### DETAILED DESCRIPTION OF THE INVENTION

### Definitions

As used herein, the following terms have the meanings ascribed to them below unless otherwise specified.

“P167” refers to a polypeptide that is at least 80% identical to a polypeptide encoded by SEQ ID NO: 1.

“P25Hh4” refers to a polypeptide that is at least 80% identical to a polypeptide encoded by SEQ ID NO: 12.

“P17Hb48” refers to a polypeptide that is at least 80% identical to a polypeptide encoded by SEQ ID NO: 15.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated P167 nucleic acid is separated from open reading frames that flank the P167 gene and encode proteins other than P167, an isolated P17Hb48 nucleic acid is separated from open reading frames that flank the P17Hb48 gene and encode proteins other than P17Hb48, and an isolated P25Hh4 nucleic acid is separated from open reading frames that flank the P25Hh4 gene and encode proteins other than P25Hh4. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

Macromolecular structures such as polypeptide structures can be described in terms of various levels of organization. For a general discussion of this organization, see, e.g., Alberts et al, Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or “detectable label” is a composition detectable by spectroscopic, photochemical, biochemical, immunochemical, or chemical means. For example, useful labels include radioisotopes (e.g., ³H, ³⁵S, ³²P, ⁵¹Cr, or ¹²⁵I), fluorescent dyes, electron-dense reagents, enzymes (e.g., alkaline phosphatase, horseradish peroxidase, or others commonly used in an ELISA), biotin, digoxigenin, or haptens and proteins for which antisera or monoclonal antibodies are available (e.g., the polypeptide encoded by SEQ ID NO: 1, SEQ ID NO:12, or SEQ ID NO:15 can be made detectable, e.g., by incorporating a radiolabel into the peptide, and used to detect antibodies specifically reactive with the peptide).

An “amplification reaction” refers to any chemical reaction, including an enzymatic reaction, which results in increased copies of a template nucleic acid sequence. Amplification reactions include polymerase chain reaction (PCR) and ligase chain reaction (LCR) (see U.S. Pat. Nos. 4,683,195 and 4,683,202; PCR Protocols: A Guide to Methods and Applications (Innis et al., eds, 1990)), strand displacement amplification (SDA) (Walker, et al. Nucleic Acids Res. 20(7):1691 (1992); Walker PCR Methods Appl 3(1):1 (1993)), transcription-mediated amplification (Phyffer, et al., J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), rolling circle amplification (RCA) (Lisby, Mol. Biotechnol. 12(1):75 (1999)); Hatch et al., Genet. Anal. 15(2):35 (1999)) and branched DNA signal amplification (bDNA) (see, e.g., Iqbal et al., Mol. Cell Probes 13(4):315 (1999)).

“Amplifying” refers to submitting a solution to conditions sufficient to allow for amplification of a polynucleotide if all of the components of the reaction are intact. Components of an amplification reaction include, e.g., primers, a polynucleotide template, polymerase, nucleotides, and the like. Thus, an amplifying step can occur without producing a product if, for example, primers are degraded.

“Amplification reagents” refer to reagents used in an amplification reaction. These reagents can include, e.g., oligonucleotide primers; borate, phosphate, carbonate, barbital, Tris, etc. based buffers (see, U.S. Pat. No. 5,508,178); salts such as potassium or sodium chloride; magnesium; deoxynucleotide triphosphates (dNTPs); a nucleic acid polymerase such as Taq DNA polymerase; as well as DMSO; and stabilizing agents such as gelatin, bovine serum albumin, and non-ionic detergents (e.g. Tween-20).

The term “primer” refers to a nucleic acid sequence that primes the synthesis of a polynucleotide in an amplification reaction. Typically a primer comprises fewer than about 100 nucleotides and preferably comprises fewer than about 30 nucleotides. Exemplary primers range from about 5 to about 25 nucleotides. The “integrity” of a primer refers to the ability of the primer to primer an amplification reaction. For example, the integrity of a primer is typically no longer intact after degradation of the primer sequences such as by endonuclease cleavage.

The term “subsequence” refers to a sequence of nucleotides that are contiguous within a second sequence but does not include all of the nucleotides of the second sequence.

A “target” or “target sequence” refers to a single or double stranded polynucleotide sequence sought to be amplified in an amplification reaction. Two target sequences are different if they comprise non-identical polynucleotide sequences.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “promoter” and “expression control sequence” are used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region such as nucleotides 1645-2799 of SEQ ID NO: 1 or nucleotides 2560-3675 of SEQ ID NO:1), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to P167 nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display. technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

An “immunogenic fragment” is one that elicits or modulates an immune response, preferably the composition induces or enhances an immune response in response to a particular P167 or a portion thereof, a particular P17Hb48 or a portion thereof, or a particular P25Hh4 or a portion thereof. Immune responses include humoral immune responses and cell-mediated immune responses, such as antibody production.

An “anti-P167” antibody is an antibody or antibody fragment that specifically binds a polypeptide encoded by a P167 gene, cDNA, or a subsequence thereof, e.g., P167A, P167B, P167C, P167D, or P167E.

An “anti-P17Hb48” antibody is an antibody or antibody fragment that specifically binds a polypeptide encoded by a P17Hb48 gene, cDNA, or a subsequence thereof.

An “anti-P25Hh4” antibody is an antibody or antibody fragment that specifically binds a polypeptide encoded by a P25Hh4 gene, cDNA, or a subsequence thereof.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “immunoassay” is an assay that uses an antibody to specifically bind an antigen. The immunoassay is characterized by the use of specific binding properties of a particular antibody to isolate, target, detect, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to P167, P17Hb48, or P25Hh4 can be selected to obtain only those polyclonal antibodies that are specifically immunoreactive with P167, P17Hb48, or P25Hh4 family members and not with other proteins. This selection may be achieved by subtracting out antibodies that cross-react with molecules such as P167, P17Hb48, or P25Hh4 from other species. In addition, polyclonal antibodies raised to P167, P17Hb48, or P25Hh4 polymorphic variants, alleles, orthologs, and conservatively modified variants can be selected to obtain only those antibodies that recognize specific fragments of P167, P17Hb48, or P25Hh4. For example polyclonal antibodies raised to can be selected to obtain only those antibodies that recognize P167 P167C or P167D, but not other P167 fragments. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind to a protein, as defined above.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

“Biological sample” as used herein is a sample of biological tissue or fluid that is suspected of containing P167, P17Hb48, or P25Hh4 polypeptides or nucleic acid encoding a P167, P17Hb48, or P25Hh4 polypeptide. These samples can be tested by the methods described herein and include animal and human body fluids such as whole blood, serum, plasma, cerebrospinal fluid, urine, lymph fluids, and various external secretions of the respiratory, intestinal and genitourinary tracts, tears, saliva, milk, white blood cells, myelomas, and the like; and biological fluids such as cell extracts, cell culture supernatants; fixed tissue specimens; and fixed cell specimens. Biological samples may also include sections of tissues such as biopsy and autopsy samples or frozen sections taken for histologic purposes. These samples are well known in the art. A biological sample is obtained from any living organism including viruses, prokaryotes or eukaryotes. A biological sample can be from a laboratory source or from a non-laboratory source. A biological sample may be suspended or dissolved in liquid materials such as buffers, extractants, solvents and the like.

I. Introduction

The present invention provides isolated polynucleotides encoding the Helicobacter antigens P167, P17Hb48, and P25Hh4. The present invention also provides polypeptides encoded by SEQ ID NO:1, SEQ ID NO:12, SEQ ID NO:15, or subsequences thereof. The present invention further provides methods and kits for more sensitive and specific detection and diagnosis of Helicobacter infections using the polynucleotides and polypeptides described herein. For example, immunoassays to detect antibodies to P167, P17Hb48, or P25Hh4 proteins or immunogenic fragments thereof can be used to qualitatively or quantitatively analyze the antibody titer for P167, P17Hb48, or P25Hh4 specific antibodies. A general overview of the applicable technology can be found in Harlow & Lane, Antibodies: A Laboratory Manual (1988). As an alternative to immunoassays, PCR amplification of nucleic acids encoding P167, P17Hb48, or P25Hh4 can be used to detect Helicobacter infection. A general overview of the applicable technology can be found in Innis et al. eds., PCR Protocols: A Guide to Methods and Applications (1990).

The P167, P17Hb48, or P25Hh4 polynucleotides, polypeptides, methods, and kits of the present invention can conveniently be used to more specifically and sensitively diagnose and monitor Helicobacter associated disease. In addition, P167, P17Hb48, or P25Hh4 or their homologues in other Helicobacter species are also useful for developing a mouse model for therapy for Helicobacter associated diseases. For example, antibodies that react specifically with P167, P17Hb48, or P25Hh4, their homologues, or immunogenic fragments thereof can be measured to monitor Helicobacter associated disease in animals, such as, for example, mice, rats, and guinea pigs. Moreover, P167, P17Hb48, or P25Hh4 protein or their homologues can conveniently be used as a target for treatment of Helicobacter associated disease.

II. Nucleic Acids Encoding P167, P17Hb48, or P25Hh4

A. General Recombinant DNA Methods

This invention relies on routine techniques in the field of recombinant genetics. Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

B. Cloning Methods for the Isolation of Nucleotide Sequences Encoding P167, P17Hb48, or P25Hh4

In general, the nucleic acid sequences encoding P167, P17Hb48, or P25Hh4 and related nucleic acid sequence homologues are cloned from cDNA and genomic DNA libraries or isolated using amplification techniques with oligonucleotide primers. For example, P167, P17Hb48, or P25Hh4 sequences are typically isolated from nucleic acid (genomic or cDNA) libraries by hybridizing with a nucleic acid probe, the sequence of which can be derived from SEQ ID NO:1, SEQ ID NO:12, SEQ ID NO:15, or a subsequence thereof, preferably from nucleotides 1645-2799 of SEQ ID NO:1 or nucleotides 2560-3675 of SEQ ID NO:1. P167 and P17Hb48 RNA and cDNA can be isolated from H. bilis. P25Hh4 RNA and cDNA can be isolated from H. hepaticus.

Nucleic acids encoding P167, P17Hb48, or P25Hh4 can also be isolated from expression libraries using antibodies as probes. Such polyclonal or monoclonal antibodies can be raised using, for example, the polypeptides encoded by the sequence of SEQ ID NO: 1, SEQ ID NO: 12, SEQ ID NO: 15, nucleotides 25-1122 of SEQ ID NO: 1, nucleotides 856-1962 of SEQ ID NO: 1, nucleotides 1645-2799 of SEQ ID NO: 1, nucleotides 2560-3675 of SEQ ID NO: 1, or nucleotides 3409-4632 of SEQ ID NO: 1.

P167, P17Hb48, or P25Hh4 polymorphic variants, alleles, and interspecies homologues that are substantially identical to P167, P17Hb48, or P25Hh4 can be isolated using P167, P17Hb48, or P25Hh4 nucleic acid probes and oligonucleotides under stringent hybridization conditions, by screening libraries. Alternatively, expression libraries can be used to clone P167, P17Hb48, or P25Hh4 polymorphic variants, alleles, and interspecies homologues, by detecting expressed homologues immunologically with antisera or purified antibodies made against the core domain of P167, P17Hb48, or P25Hh4 which also recognize and selectively bind to the P167, P17Hb48, or P25Hh4 homologue.

To make a cDNA library, P167 and P17Hb48 mRNA may be purified from H. bilis and P25Hh4 mRNA may be purified from H. hepaticus. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 1-8 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating P167, P17Hb48, or P25Hh4 nucleic acids and their homologues combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of P167, P17Hb48, or P25Hh4 directly from mRNA, from cDNA, from genomic libraries or cDNA libraries. Degenerate oligonucleotides can be designed to amplify P167, P17Hb48, or P25Hh4 homologues using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of P167, P17Hb48, or P25Hh4 encoding mRNA in physiological samples, for nucleic acid sequencing, or for other purposes. Genes amplified by the PCR reaction can be purified from agarose gels and cloned into an appropriate vector.

Amplification techniques using primers can also be used to amplify and isolate P167, P17Hb48, or P25Hh4 DNA or RNA. For example, nucleic acids encoding P167 or fragments thereof may be obtained by amplification of a H. bilis cDNA library or reverse transcribed from H. bilis RNA using isolated nucleic acid primer pairs having the following sequences:

| | | | |
|---|---|---|---|
| P167A | 5′ primer: | TATGCTGGGGATATTCAAGGCGAT and | (SEQ ID NO:2) |
| | 3′ primer | ATTGACATGTATCCAGCTACC; | (SEQ ID NO:3) |
| P167B | 5′ primer: | ATAGATGATGGCAGTAGCACC and | (SEQ ID NO:4) |
| | 3′ primer | TGCTATCTCATCACCACTCAT; | (SEQ ID NO:5) |
| P167C | 5′ primer | CGTATGGGTGAGATTAAGCATGTC and | (SEQ ID NO:6) |
| | 3′ primer | TGAGCCTATGCCATTTTCTACTAC; | (SEQ ID NO:7) |
| P167D | 5′ primer: | AGCTACACTTACACACAAGGGGAT and | (SEQ ID NO:8) |
| | 3′ primer: | ATCTGTTACTCCATTGTTTGC; | (SEQ ID NO:9) |
| P167E | 5′ primer: | CGTCTAGCAAGAGTAGCTAGCATT and | (SEQ ID NO:10) |
| | 3′ primer: | ATTGGTGGTAGGGTTGTGTTTTAG. | (SEQ ID NO:11) |

Nucleic acids encoding P17Hb48 or fragments thereof may be obtained by amplification of a H. bilis cDNA library or reverse transcribed from H. bilis RNA using isolated nucleic acid primer pairs having the following sequences: P17Hb48 5′primer:

| | | | |
|---|---|---|---|
| P17Hb48 | 5′primer: | ATGGAACAGATAAAGATTTTAAAGCAACTTCAG | (SEQ ID NO:16) |
| and | | | |
| | 3′primer: | CTATGCAAGTTGTGCGTTAAGCAT | (SEQ ID NO:17) |

Nucleic acids encoding P25Hh4 or fragments thereof may be obtained by amplification of a H. hepaticus cDNA library or reverse transcribed from H. hepaticus RNA using isolated nucleic acid primer pairs having the following sequences: P25Hh4 5′primer:

(SEQ ID NO:13)

| | | |
|---|---|---|
| P25Hh4 | 5′primer: | ATGGGTAAGAAAATAGCAAAAAGATTGCAA |
| and | | |

(SEQ ID NO:14)

| | |
|---|---|
| 3′primer: | CTATTTCATATCCATAAGCTCTTGAGAATC. |

These primers can be used, e.g., to amplify either the full length sequence or a probe of one to several hundred nucleotides, which is then used to screen a cDNA library for full-length P167, P17Hb48, or P25Hh4.

Gene expression of P167, P17Hb48, or P25Hh4 can also be analyzed by techniques known in the art, e.g., reverse transcription and amplification of mRNA, isolation of total RNA or poly A+ RNA, northern blotting, dot blotting, in situ hybridization, RNase protection, probing DNA microchip arrays, and the like.

Synthetic oligonucleotides can be used to construct recombinant P167, P17Hb48, or P25Hh4 genes for use as probes or for expression of protein. This method is performed using a series of overlapping oligonucleotides usually 40-120 bp in length, representing both the sense and non-sense strands of the gene. These DNA fragments are then annealed, ligated and cloned. Alternatively, amplification techniques can be used with precise primers to amplify a specific subsequence of the P167, P17Hb48, or P25Hh4 gene. The specific subsequence is then ligated into an expression vector. P167, P17Hb48, or P25Hh4 chimeras can be made, which combine, e.g., a portion of P167, P17Hb48, or P25Hh4 with a portion of a heterologous P167, P17Hb48, or P25Hh4 to create a chimeric, functional P167, P17Hb48, or P25Hh4.

The gene for P167, P17Hb48, or P25Hh4 is typically cloned into intermediate vectors before transformation into prokaryotic or eukaryotic cells for replication and/or expression. These intermediate vectors are typically prokaryote vectors, e.g., plasmids, or shuttle vectors. Isolated nucleic acids encoding P167 proteins comprise a nucleic acid sequence (SEQ ID NO:1, SEQ ID NO:12, or SEQ ID NO:15) encoding a P167 protein and subsequences, interspecies homologues, alleles and polymorphic variants thereof. In preferred embodiments, the isolated nucleic acid encoding a P167 protein is SEQ ID NO:1, nucleotides 25-1122 of SEQ ID NO:1, nucleotides 856-1962 of SEQ ID NO:1, nucleotides 1645-2799 of SEQ ID NO: 1, nucleotides 2560-3675 of SEQ ID NO: 1, nucleotides 3409-4632 of SEQ ID NO: 1 or complements thereof.

C. Expression of P167, P17Hb48, or P25Hh14 in Prokaryotes and Eukaryotes

To obtain high level expression of a cloned gene, such as those cDNAs encoding P167, P17Hb48, or P25Hh4, one typically subclones P167, P17Hb48, or P25Hh4 into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al. Bacterial expression systems for expressing the P167, P17Hb48, or P25Hh4 protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the P167, P17Hb48, or P25Hh4 encoding nucleic acid in host cells. A typical expression cassette thus contains a promoter operably linked to the nucleic acid sequence encoding P167, P17Hb48, or P25Hh4 and signals required for efficient polyadenylation of the transcript, ribosome binding sites, and translation termination. The nucleic acid sequence encoding P167, P17Hb48, or P25Hh4 may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a P167, P17Hb48, or P25Hh4 encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of P167, P17Hb48, or P25Hh4 protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing P167, P17Hb48, or P25Hh4.

After the expression vector is introduced into the cells, the transfected cells are cultured under conditions favoring expression of P167, P17Hb48, or P25Hh4, which is recovered from the culture using standard techniques identified below.

D. Purification of P167, P17Hb48, or P25Hh4 Protein

Either naturally occurring or recombinant P167, P17Hb48, or P25Hh4 can be purified for use in functional assays. Naturally occurring P167 or P 17Hb48 are purified, e.g., from H. bilis and any other source of a P167 homologue, such as, for example, H. muridarum, H. pylori, H. rodentium, H. typhlonicus, or H. hepaticus. Naturally occurring P25Hh4 is purified, e.g., from H. hepaticus and any other source of a P25Hh4 homologue, such as, for example, H. muridarum, H. pylori, H. rodentium, H. typhlonicus, or H. bilis. Recombinant P167, P17Hb48, or P25Hh4 is purified from any suitable expression system.

P167, P17Hb48, or P25Hh4 may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al, supra; and Sambrook et al., supra).

A number of procedures can be employed when recombinant P167, P17Hb48, or P25Hh4 is being purified. For example, proteins having established molecular adhesion properties can be reversible fused to P167, P17Hb48, or P25Hh4. With the appropriate ligand, P167, P17Hb48, or P25Hh4 can be selectively adsorbed to a purification column and then freed from the column in a relatively pure form. The fused protein is then removed by enzymatic activity. Finally P167, P17Hb48, or P25Hh4 could be purified using immunoaffinity columns.

Purification of P167, P17Hb48, or P25Hh4 from Recombinant Bacteria

Recombinant proteins are expressed by transformed bacteria in large amounts, typically after promoter induction; but expression can be constitutive. Promoter induction with IPTG is a one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of P167, P17Hb48, or P25Hh4 inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. P167, P17Hb48, or P25Hh4 is separated from other bacterial proteins by standard separation techniques, e.g., with Ni-NTA agarose resin.

Alternatively, it is possible to purify P167, P17Hb48, or P25Hh4 from bacteria periplasm. After lysis of the bacteria, when P167, P17Hb48, or P25Hh4 is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying P167, P17Hb48, or P25Hh4

Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of P167, P17Hb48, or P25Hh4 can be used to isolated it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

P167, P17Hb48, or P25Hh4 can also be separated from other proteins on the basis of their size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Alternatively, P167, P17Hb48, or P25Hh4 protein can be expressed transiently in a cell by introducing into a cell an RNA encoding the P167, P17Hb48, or P25Hh4 protein. The RNA is transcribed in vitro according to standard procedures and then introduced into a cell (e.g. such as Xenopus oocytes, CHO, and HeLa cells) by means such as injection or electroporation. The RNA then expresses the P167, P17Hb48, or P25Hh4 protein.

III. Immunological Detection of Helicobacter Infection

In addition to the detection of Helicobacter infection by detaction of P167, P17Hb48, or P25Hh4 genes and gene expression using nucleic acid hybridization technology, one can also use immunoassays to detect Helicobacter infection by detecting P167, P17Hb48, or P25Hh4 or antibodies that specifically bind to them. Immunoassays can be used to qualitatively or quantitatively analyze P167, P17Hb48, or P25Hh4. A general overview of the applicable technology can be found in Harlow & Lane, Antibodies: A Laboratory Manual (1988).

A. Antibodies to P167, P17Hb48, or P25Hh4

Methods of producing polyclonal and monoclonal antibodies that react specifically with P167, P17Hb48, or P25Hh4, or immunogenic fragments of P167, such as, P167A, P167B, P167C, P167D, or P167E, or immunogenic fragments of P17Hb48 or P25Hh4, are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A number of immunogens comprising portions of P167, P17Hb48, or P25Hh4 may be used to produce antibodies specifically reactive with P167, P17Hb48, or P25Hh4 or homologues thereof. For example, recombinant P167C (encoded by nucleotides 1645-2799 of SEQ ID NO: 1) or P167D (encoded by nucleotides 2560-3675 of SEQ ID NO: 1) or antigenic fragment thereof, can be isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-P167, non-P17Hb48, or non-P25Hh4 proteins and P167, P17Hb48, or P25Hh4 proteins, using a competitive binding immunoassay. Specific polyclonal antisera and monoclonal antibodies will usually bind with a Kd of at least about 0.1 mM, more usually at least about 1 μM, preferably at least about 0.1 μM or better, and most preferably, 0.01 μM or better. Antibodies specific only for a particular P167, P17Hb48, or P25Hh4 homologue, such as H. pylori P167, P17Hb48, or P25Hh4, can also be made, by subtracting out other cross-reacting homologues from a species such as a non-human mammal.

Once the specific antibodies against a P167, P17Hb48, or P25Hh4 are available, P167, P17Hb48, or P25Hh4 homologues can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra. Additional assay configurations (i.e., using multiplex assays using microspheres) are described in, e.g., De Jager et al., Clin. Diagn. Lab. Immunol. 10(1):13309 (2003); Earley et al., Cytometry 50(5):239-42 (2002); and Seidman and Peritt, J. Immunol. Methods 267(2):165-71 (2002).

In one exemplary embodiment, the immunoassays are performed using Luminex technology. With Luminex technology, molecular reactions take place on the surface of microscopic beads called microspheres (Literature from Luminex Corporation, Austin, Tex.). For each reaction in a Luminex profile, thousands of molecules are attached to the surface of internally color-coded microspheres. The assigned color-code identifies each reaction throughout the test. The magnitude of the biomolecular reaction is measured using a second molecule called a reporter which can be a secondary antibody labeled with color. The reporter molecule signals the extent of the reaction by attaching to the molecules on the microspheres. Because the reporter's signal is also a color, there are two sources of color, the color-code inside the microsphere and the reporter color on the surface of the microsphere. To perform a test, the color-coded microspheres, reporter molecules, and sample are combined. This mixture is then injected into an instrument that uses microfluidics to align the microspheres in single file where lasers illuminate the colors inside and on the surface of each microsphere. Next, advanced optics capture the color signals. Finally, digital signal processing translates the signals into real-time, quantitative data for each reaction. The advantages of this Luminex techniques are that multiplex antigens representing different pathogens can be tested with single serum sample, therefore, it saves on labor, reagents, time and samples: and that it makes high throughput (20,000 microsphere per second) possible and shortens analysis time. For example, one color coded beads can be coated with P167, and a different color coded beads can be coated with P25Hh4: and 2 sets of beads can be mixed and reacted with the same fluid sample to determine whether the sample has H. bilis, or H. hepaticus, or both by a single test.

B. Immunological Binding Assays

The P167, P17Hb48, or P25Hh4 polypeptides of the invention and antibodies that specifically bind to them can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case P167, P17Hb48, P25Hh4 or an immunogenic fragment thereof). The antibody (e.g., anti-P167, anti-P 17Hb48, or anti-P25Hh4) may be produced by any of a number of means well known to those of skill in the art and as described above. Alternatively, a protein or antigen of choice (in this case P167, P17Hb48, P25Hh4, or an immunogenic fragment thereof) may be used to bind antibodies that specifically bind to the protein or antigen. The protein or antigen may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled P167, P17Hb48, or P25Hh4 polypeptide or a labeled anti-P167, anti-P17Hb48, or anti-P25Hh4 antibody. Alternatively, the labeling agent may be a third moiety, such a secondary antibody, which specifically binds to the antibody/P167 complex, the antibody/P17Hb48 complex, or the antibody/P25Hh4 complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111:1401-1406 (1973); Akerstrom et al., J. Immunol. 135:2589-2542 (1985)). The labeling agent can be modified with a detectable moiety, such as biotin, to which another molecule can specifically bind, such as streptavidin. The streptavidin may be bound to a label or detectable group as discussed below. A variety of detectable moieties are well known to those skilled in the art.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADSTM), fluorescent dyes (e.g., fluorescein isothiocyanate, Texas red, rhodamine, and the like), radiolabels (e.g., ³H, ¹²⁵I, ³⁵S, ¹4C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecule (e.g., streptavidin), which is either inherently detectable or covalently bound to a signal system, such as a detectable enzyme, a fluorescent compound, or a chemiluminescent compound. The ligands and their targets can be used in any suitable combination with antibodies that recognize P167, P17Hb48, or P25Hh4, or secondary antibodies that recognize anti-P167, anti-P17Hb48, or anti-P25Hh4 antibodies.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Non-Competitive Assay Formats

Immunoassays for detecting P167, P17Hb48, or P25Hh4 or immunogenic fragments thereof in samples may be either competitive or noncompetitive. Noncompetitive immunoassays are assays in which the amount of antigen is directly measured. In one preferred “sandwich” assay, for example, the anti-P167, anti-P17Hb48, or anti-P25Hh4 antibodies can be bound directly to a solid substrate on which they are immobilized. These immobilized antibodies then capture P167, P17Hb48, or P25Hh4 present in the test sample. P167, P17Hb48, or P25Hh4 are thus immobilized and then bound by a labeling agent, such as a second P167 antibody, P17Hb48 antibody, or P25Hh4 antibody bearing a label. Alternatively, the second antibody may lack a label, but it may, in turn, be bound by a labeled third antibody specific to antibodies of the species from which the second antibody is derived. The second or third antibody is typically modified with a detectable label, such as biotin, to which another molecule specifically binds, e.g., streptavidin, to provide a detectable moiety.

Noncompetitive immunoassays may also be assays in which the amount of anti-P167, anti-P17Hb48, or anti-P25Hh4 antibody is directly measured. P167, P17Hb48, or P25Hh4 or an immunogenic fragment thereof can be bound directly to a solid substrate on which they are immobilized. The immobilized P167, P17Hb48, or P25Hh4 then captures anti-P167, anti-P17Hb48, or anti-P25Hh4 antibodies present in the test sample. Anti-P167, anti-P17Hb48, or anti-P25Hh4 antibodies are thus immobilized and then bound by a labeling agent, such as an anti-Fc antibody bearing a label. The anti-Fc antibody may be, for example, an anti-mouse Fc antibody, an anti-rat Fc antibody, or an anti-rabbit Fc antibody. Those of skill in the art will appreciate that any suitable anti-Fc antibody may be selected for use in this type of assay. Alternatively, the anti-Fc antibody may lack a label, but it may, in turn, be bound by a labeled third antibody specific to antibodies of the species from which the second antibody is derived. The second or third antibody is typically modified with a detectable label, such as biotin, to which another molecule specifically binds, e.g., streptavidin, to provide a detectable moiety.

Competitive Assay Formats

In competitive assays, the amount of the P167, P17Hb48, or P25Hh4 present in the sample is measured indirectly by measuring the amount of known, added (exogenous) P167, P17Hb48, or P25Hh4 displaced (competed away) from an anti-P167, anti-P17Hb48, or anti-P25Hh4 antibody by the unknown P167, P17Hb48, or P25Hh4 present in a sample. In one competitive assay, a known amount of the P167, P17Hb48, or P25Hh4 is added to a sample and the sample is then contacted with an antibody that specifically binds to the P167, P17Hb48, or P25Hh4. The amount of exogenous P167, P17Hb48, or P25Hh4 bound to the antibody is inversely proportional to the concentration of the P167, P17Hb48, or P25Hh4 present in the sample. In a particularly preferred embodiment, the antibody is immobilized on a solid substrate. The amount of P167, P17Hb48, or P25Hh4 bound to the antibody may be determined either by measuring the amount of P167, P17Hb48, or P25Hh4 present in a P167/antibody complex, P17Hb48/antibody complex, P25Hh4/antibody complex, or alternatively by measuring the amount of remaining uncomplexed protein. The amount of P167, P17Hb48, or P25Hh4 may be detected by providing a labeled P167, labeled P17Hb48, or labeled P25Hh4 molecule.

A hapten inhibition assay is another preferred competitive assay. In this assay the known P167, P17Hb48, or P25Hh4 is immobilized on a solid substrate. A known amount of anti-P167, anti-P17Hb48, or anti-P25Hh4 antibody is added to the sample, and the sample is then contacted with the immobilized P167, P17Hb48, or P25Hh4. The amount of anti-P167, anti-P17Hb48, or anti-P25Hh4 antibody bound to the known immobilized P167, P17Hb48, or P25Hh4 is inversely proportional to the amount of P167, P17Hb48, or P25Hh4 present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Cross-Reactivity Determinations

Immunoassays in the competitive binding format can also be used for crossreactivity determinations for P167, P17Hb48, or P25Hh4 homologues. For example, a P167 protein at least partially corresponding to a polypeptide sequence encoded by SEQ ID NO:1 or an immunogenic fragment thereof, such, e.g., a polypeptide encoded by nucleotides 1645-2799 of SEQ ID NO: 1 or a polypeptide encoded by nucleotides 2560-3675 of SEQ ID NO: 1), a P17Hb48 protein at least partially corresponding to a polypeptide sequence encoded by SEQ ID NO: 12 or an immunogenic fragment thereof, a P25Hh4 protein at least partially corresponding to a polypeptide sequence encoded by SEQ ID NO: 15 or an immunogenic fragment thereof, can be immobilized to a solid support. Other proteins such as P167, P17Hb48, or P25Hh4 homologues or other proteins from other Helicobacter species such as, for example, H. hepaticus, H. muridarum, H. pylori, H. typhlonicus, H. rodentium, or Flexispira rappini, are added to the assay so as to compete for binding of the antisera to the immobilized antigen. The ability of the added proteins to compete for binding of the antisera to the immobilized protein is compared to the ability of the P167, P 17Hb48, or P25Hh4 or immunogenic portion thereof to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologues. Antibodies that specifically bind only to P167, P17Hb48, or P25Hh4, or only to particular homologues of P167, P17Hb48, or P25Hh4, such as P167 or P17Hb48 from H. bilis, or P25Hh4 from H. hepaticus can also be made using this methodology.

The immunoabsorbed and pooled antisera are then used in a competitive binding immunoassay as described above to compare a second protein, thought to be perhaps a P167, P17Hb48, or P25Hh4 homologue or an allele, or polymorphic variant of P167, P17Hb48, or P25Hh4, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein encoded by P167, P17Hb48, or P25Hh4 that is required to inhibit 50% of binding, then the second protein is said to specifically bind to the polyclonal antibodies generated to the respective P167, P17Hb48, or P25Hh4 immunogen.

Other Assay Formats

Western blot (immunoblot) analysis is used to detect and quantify the presence of the P167, P17Hb48, or P25Hh4 polypeptides in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind P167, P17Hb48, or P25Hh4 polypeptides. The anti-P167, anti-P17Hb48, or anti-P25Hh4 antibodies specifically bind to P167, P17Hb48, or P25Hh4 on the solid support, thereby forming an antibody-polypeptide complex. These antibodies may be directly labeled or alternatively may be subsequently detected using labeled antibodies (e.g., labeled sheep anti-mouse antibodies) that specifically bind to the anti-P167, anti-P17Hb48, or anti-P25Hh4 antibodies.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev. 5:34-41 (1986)).

Reduction of Non-Specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

IV. PCR Detection of Helicobacter Infection

In addition to the detection of P167, P17Hb48, or P25Hh4 genes and gene expression immunoassays PCR can be used to detect Helicobacter infection by amplification of nucleic acids encoding P167, P17Hb48, or P25Hh4. A general overview of the applicable technology can be found in PCR Protocols: A Guide to Methods and Applications (Innis et al. eds. (1990)) and PCR Technology: Principles and Applications for DNA Amplification (Erlich, ed. (1992)).

PCR permits the copying, and resultant amplification of a target nucleic acid, e.g., a nucleic acid encoding P167, P17Hb48, or P25Hh4. Briefly, a target nucleic acid, e.g. DNA from a biological sample from a subject suspected of having a Helicobacter infection, is combined with a sense and antisense primers, dNTPs, DNA polymerase and other reaction components. (See, Innis et al., supra) The sense primer can anneal to the antisense strand of a DNA sequence of interest. The antisense primer can anneal to the sense strand of the DNA sequence, downstream of the location where the sense primer anneals to the DNA target. In the first round of amplification, the DNA polymerase extends the antisense and sense primers that are annealed to the target nucleic acid. The first strands are synthesized as long strands of indiscriminate length. In the second round of amplification, the antisense and sense primers anneal to the parent target nucleic acid and to the complementary sequences on the long strands. The DNA polymerase then extends the annealed primers to form strands of discrete length that are complementary to each other. The subsequent rounds serve to predominantly amplify the DNA molecules of the discrete length.

In general, PCR and other methods of amplification use primers which anneal to either end of the DNA of interest. For example, nucleic acids encoding P167 or fragments thereof may be amplified using isolated nucleic acid primer pairs having the following sequences: P167A 5′ primer: TATGCTGGGGATATTCAAGGCGAT (SEQ ID NO:2) and 3′ primer ATTGACATGTATCCAGCTACC (SEQ ID NO:3); P167B 5′ primer: ATAGATGATGGCAGTAGCACC (SEQ ID NO:4) and 3′ primer TGCTATCTCATCACCACTCAT (SEQ ID NO:5); P167C 5′ primer CGTATGGGTGAGATTAAGCATGTC (SEQ ID NO:6) and 3′ primer TGAGCCTATGCCATTTTCTACTAC (SEQ ID NO:7); P167D 5′ primer: AGCTACACTTACACACAAGGGGAT (SEQ ID NO:8) and 3′ primer: ATCTGTTACTCCATTGTTTGC (SEQ ID NO:9); P167E 5′ primer: CGTCTAGCAAGAGTAGCTAGCATT (SEQ ID NO:10) and 3′ primer: ATTGGTGGTAGGGTTGTGTTTTAG (SEQ ID NO: 11). Amplification of DNA encoding P167 from a biological sample from a subject suspected of having a Helicobacter infection indicates that the subject has a Helicobacter bilis infection.

Nucleic acids encoding P17Hb48 or fragments thereof may be identified by amplification using isolated nucleic acid primer pairs having the following sequences: P17Hb48 5′primer: ATGGAACAGATAAAGATTTTAAAGCAACTTCAG (SEQ ID NO:16) and 3′primer: CTATGCAAGTTGTGCGTTAAGCAT (SEQ ID NO: 17). Amplification of DNA encoding P17Hb48 from a biological sample from a subject suspected of having a Helicobacter infection indicates that the subject has a Helicobacter bilis infection.

Nucleic acids encoding P25Hh4 or fragments thereof may be identified by amplification using isolated nucleic acid primer pairs having the following sequences: P25Hh4 5′primer: ATGGGTAAGAAAATAGCAAAAAGATTGCAA (SEQ ID NO:13) and 3′primer: CTATTTCATATCCATAAGCTCTTGAGAATC (SEQ ID NO: 14). Amplification of DNA encoding P25Hh4 from a biological sample from a subject suspected of having a Helicobacter infection indicates that the subject has a Helicobacter hepaticus infection.

Target nucleic acid sequences may be double or single-stranded DNA or RNA from any biological source suspected of having a Helicobacter infection. Preferably, the target template is an isolated DNA sequence. Target DNA sequences may be isolated using a variety of techniques. For example, methods are known for lysing organisms and preparing extracts or purifying DNA. See, Current Protocols in Molecular Biology Volumes 1-3, John Wiley & Sons, Inc. (Ausubel et al., eds., 1994-1998) (hereinafter “Ausubel et al.”). Also, total RNA or polyA+ RNA can be reverse transcribed to produce cDNA that can serve as the target DNA.

A. Reaction Components

1. Oligonucleotides

The oligonucleotides that are used in the present invention as well as oligonucleotides designed to detect amplification products can be chemically synthesized, as described above. These oligonucleotides can be labeled with radioisotopes, chemiluminescent moieties, or fluorescent moieties. Such labels are useful for the characterization and detection of amplification products using the methods and compositions of the present invention.

The primer components may be present in the PCR reaction mixture at a concentration of, e.g., between 0.1 and 1.0 μM. The concentration of the target primers can be from about 0.1 to about 0.75 μM. The primer length can be between, e.g., 15-100 nucleotides in length and preferably have 40-60% G and C composition. In the choice of primer, it is preferable to have exactly matching bases at the 3′ end of the primer but this requirement decreases to relatively insignificance at the 5′ end. Preferably, the primers of the invention all have approximately the same melting temperature.

Typically, the primers have the following design. The most 3′ portion anneals to the constant region flanking the target region to be amplified, this portion will normally have at least 6 bp of homology to the target region, preferably 9 or more bp. The region of homology is adjacent to the restriction enzyme sequence. If this recognition site is an interrupted sequence, the intervening portion of sequence between the two portions of the restriction enzyme site will normally contain bases which can anneal to the appropriate portion of the constant region flanking the target of interest. 5′ to the restriction enzyme site are sufficient bases to allow the restriction enzyme to recognize its site and cleave the recognized sequence. Where the restriction enzyme site cleaves twice, once on either side of the recognition site, the primer should be sufficiently long to allow the enzyme to cleave at both of the cleavage sites. The extra nucleotides may or may not have further homology to the constant region flanking the target of interest.

2. Buffer

Buffers that may be employed are borate, phosphate, carbonate, barbital, Tris, etc. based buffers. (See, U.S. Pat. No. 5,508,178). The pH of the reaction should be maintained in the range of about 4.5 to about 9.5. (See, U.S. Pat. No. 5,508,178. The standard buffer used in amplification reactions is a Tris based buffer between 10 and 50 mM with a pH of around 8.3 to 8.8. (See Innis et al., supra.).

One of skill in the art will recognize that buffer conditions should be designed to allow for the function of all reactions of interest. Thus, buffer conditions can be designed to support the amplification reaction as well as any subsequent restriction enzyme reactions. A particular reaction buffer can be tested for its ability to support various reactions by testing the reactions both individually and in combination.

3. Salt Concentration

The concentration of salt present in the reaction can affect the ability of primers to anneal to the target nucleic acid. (See, Innis et al.). Potassium chloride is added up to a concentration of about 50 mM to the reaction mixture to promote primer annealing. Sodium chloride can also be added to promote primer annealing. (See, Innis et al.).

4. Magnesium Ion Concentration

The concentration of magnesium ion in the reaction can affect amplification of the target sequence(s). (See, Innis et al.). Primer annealing, strand denaturation, amplification specificity, primer-dimer formation, and enzyme activity are all examples of parameters that are affected by magnesium concentration. (See, Innis et al.). Amplification reactions should contain about a 0.5 to 2.5 mM magnesium concentration excess over the concentration of dNTPs. The presence of magnesium chelators in the reaction can affect the optimal magnesium concentration. A series of amplification reactions can be carried out over a range of magnesium concentrations to determine the optimal magnesium concentration. The optimal magnesium concentration can vary depending on the nature of the target nucleic acid(s) and the primers being used, among other parameters.

5. Deoxynucleotide Triphosphate Concentration

Deoxynucleotide triphosphates (dNTPs) are added to the reaction to a final concentration of about 20 μM to about 300 μM. Typically, each of the four dNTPs (G, A, C, T) are present at equivalent concentrations. (See, Innis et al.).

6. Nucleic Acid Polymerase

A variety of DNA dependent polymerases are commercially available that will function using the methods and compositions of the present invention. For example, Taq DNA Polymerase may be used to amplify target DNA sequences. The PCR assay may be carried out using as an enzyme component a source of thermostable DNA polymerase suitably comprising Taq DNA polymerase which may be the native enzyme purified from Thermus aquaticus and/or a genetically engineered form of the enzyme. Other commercially available polymerase enzymes include, e.g., Taq polymerases marketed by Promega or Pharmacia. Other examples of thermostable DNA polymerases that could be used in the invention include DNA polymerases obtained from, e.g., Thermus and Pyrococcus species. Concentration ranges of the polymerase may range from 1-5 units per reaction mixture. The reaction mixture is typically between 20 and 100 μl.

In some embodiments, a “hot start” polymerase can be used to prevent extension of mispriming events as the temperature of a reaction initially increases. Hot start polymerases can have, for example, heat labile adducts requiring a heat activation step (typically 95° C. for approximately 10-15 minutes) or can have an antibody associated with the polymerase to prevent activation.

7. Other Agents

Additional agents are sometime added to the reaction to achieve the desired results. For example, DMSO can be added to the reaction, but is reported to inhibit the activity of Taq DNA Polymerase. Nevertheless, DMSO has been recommended for the amplification of multiple target sequences in the same reaction. (See, Innis et al. supra). Stabilizing agents such as gelatin, bovine serum albumin, and non-ionic detergents (e.g. Tween-20) are commonly added to amplification reactions. (See, Innis et al. supra).

B. Amplification

Amplification of an RNA or DNA template using reactions is well known (see, U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of target DNA sequences (e.g., DNA encoding P167, P17Hb48, or P25Hh4) from nucleic acids purified from biological samples obtained from subjects suspected of having a Helicobacter infection. The reaction is preferably carried out in a thermal cycler to facilitate incubation times at desired temperatures. Degenerate oligonucleotides can be designed to amplify target DNA sequence homologs using the known sequences that encode the target DNA sequence, e.g., DNA encoding P167, P17Hb48, or P25Hh4. Polymerase chain reaction or other in vitro amplification methods may also be useful, for example, to clone nucleic acid sequences that code for the target DNA sequence proteins to be expressed. Genes amplified by the PCR reaction can be purified from agarose gels and cloned into an appropriate vector.

Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

V. IV. Kits

P167, P17Hb48, or P25Hh4 and their homologues are a useful tool for more specific and sensitive diagnosis and detection of Helicobacter infections, e.g., in mice and other rodents. P167, P17Hb48, or P25Hh4 specific reagents that specifically hybridize to P167, P17Hb48, or P25Hh4 nucleic acid, such as P167, P17Hb48, or P25Hh4 probes and primers, P167 polynucleotides, such as, P167A, P167B, P167C, P167D, and P167E, P17Hb48 polynucleotides, P25Hh4 polynucleotides, and P167, P17Hb48, or P25Hh4 specific reagents that specifically bind to the P167, P17Hb48, or P25Hh4 protein, e.g., P167 antibodies and P17Hb48 antibodies are used to diagnose and detect H. bilis infections and P25Hh4 antibodies are used to diagnose and detect H. hepaticus infections.

Nucleic acid assays for the presence of P167, P17Hb48, or P25Hh4 DNA and RNA in a sample include numerous techniques are known to those skilled in the art, such as Southern analysis, northern analysis, dot blots, RNase protection, S1 analysis, amplification techniques such as PCR and LCR, and in situ hybridization. In in situ hybridization, for example, the target nucleic acid is liberated from its cellular surroundings in such as to be available for hybridization within the cell while preserving the cellular morphology for subsequent interpretation and analysis. The following articles provide an overview of the art of in situ hybridization: Singer et al., Biotechniques 4:230-250 (1986); Haase et al., Methods in Virology, vol. VII, pp. 189-226 (1984); and Nucleic Acid Hybridization: A Practical Approach (Hames et al., eds. 1987). In addition, P167, P17Hb48, or P25Hh4 protein can be detected with the various immunoassay techniques described above, e.g., ELISA, western blots, etc. The test sample is typically compared to both a positive control (e.g., a sample expressing recombinant P167, P17Hb48, or P25Hh4) and a negative control. A wide variety of kits and components can be prepared according to the present invention, depending upon the intended user of the kit and the particular needs of the user. For example, the kit can be tailored for in vitro or in vivo assays.

The invention also provides kits and solutions for carrying out the amplification methods of the invention. For example, the invention provides kits that include one or more reaction vessels that have aliquots of some or all of the reaction components of the invention in them. Aliquots can be in liquid or dried form. Reaction vessels can include sample processing cartridges or other vessels that allow for the containment, processing and/or amplification of samples in the same vessel. Such kits allow for ready detection of amplification products of the invention into standard or portable amplification devices. The kits can also include written instructions for the use of the kit to amplify and control for amplification of a target sample.

Kits can include, for instance, amplification reagents comprising primers sufficient to amplify at least two different target sequences, a polynucleotide sequence comprising the sequences of the primers or subsequences of the primers s described herein; and at least one probe for amplifying and detecting the polynucleotide sequence. In addition, the kit can include nucleotides (e.g., A, C, G and T), a DNA polymerase and appropriate buffers, salts and other reagents to facilitate amplification reactions.

In some embodiments, the kits comprise vessels such as sample processing cartridges useful for rapid amplification of a sample as described in Belgrader, P., et al., Biosensors and Bioelectronics 14:849-852 (2000); Belgrader, P., et al., Science, 284:449-450 (1999); and Northrup, M. A., et al. “A New Generation of PCR Instruments and Nucleic Acid Concentration Systems” in PCR PROTOCOLS (Sninsky, J. J. et al (eds.)) Academic, San Diego, Chapter 8 (1998)).

### EXAMPLES

The following examples are offered to illustrate, but not to limit the claimed invention.

### Example 1

### Materials and Methods

Mice: Virus antibody- and Helicobacter-free C3H/HeN (C3H) and C3H/Smn.CIcrHsd-scid (C3H-scid) mice were purchased at 3-5 weeks of age from the National Cancer Institute Animal Production Program, Frederick Cancer Research Center, Frederick, Md. (C3H) or Harlan Sprague-Dawley, Indianapolis, Ind. (C3H scid). Upon arrival, fecal pellets from all mice were tested for Helicobacter by culture (below) and PCR (Hodzic, et al. (2001) Comp. Med. 51: 406-412) (Shames, et al. (1995) J. Clin. Microb. 33: 2968-2972). Mice were maintained in a pathogen-free room with restricted access on a 12:12 light cycle and fed irradiated Pico Lab Mouse Diet 20 (PMI Nutrition International, Inc., Brentwood, Mo.). Mice were killed with CO₂ narcosis. All procedures and use of mice were in compliance with the PHS Guide for the Care and Use of Laboratory Animals.

Bacterial culture and isolation: Helicobacter bilis (ATCC No. 51630), H. hepaticus (ATCC No. 51448), and H. muridarum (ATCC No. 49282) were obtained from the American Type Culture Collection and cloned by 3×limiting dilution, as described (Hodzic, et al. (2001) Comp. Med. 51: 406-412). The species identity of each clonal population was verified by PCR (Hodzic, et al. (2001) Comp. Med. 51: 406-412). To determine H. bilis infection in nice, freshly pooled fecal pellets were processed and cultured in Brucella broth, as described (Hodzic, et al. (2001) Comp. Med. 51: 406-412). Isolates were verified to be H. bilis by PCR, using H. bilis-specific 16S rDNA primers (Riley, et al. (1996) J. Clin. Microb. 34: 942-946). For DNA extraction and PCR amplification from feces, samples were processed as described (Shames, et al. (1995) J. Clin. Microb. 33: 2968-2972). Assays included negative controls from uninfected mice, and H. bilis genomic DNA served as a positive control. B. burgdorferi sensu stricto, cN40, were cultured in modified Barbour-Stoenner-Kelly (BSK II) medium at 33° C., as described (Feng, et al. (1998) Infect. Immun. 66: 2827-2835) and were used as a negative control.

Immune serum and antiserum: H. bilis was grown under microacrobic conditions in Brucella broth for three days at 37° C. as described (Hodzic, et al. (2001) Comp. Med. 51: 406-412). Bacteria were adjusted to 10⁸ colony-forming units per ml, and 0.1 ml was inoculated intraperitoneally into C3H-scid mice, as described (Hodzic, et al. (2001) Comp. Med. 51: 406-412). Once infection was established (4 to 8 weeks after inoculation) and confirmed by fecal PCR, the mice were killed and livers collected. Liver tissue containing host-adapted H. bilis was homogenized in 10 ml Brucella broth, and then 0.25 ml of the homogenate was inoculated by gavage into C3H mice. Infection status was monitored weekly by fecal PCR and culture. At 6 months after infection, blood was collected and serum harvested from positive mice.

Sera were also obtained and tested from naturally infected C3H/HeN mice. Speciation of the infecting Helicobacter was performed by fecal PCR using Helicobacter genus-specific primers (Beckwith, et al. (1997) J. Clin. Microb. 35: 1620-1623) followed by restriction enzyme digestion of PCR amplicons to differentiate H. hepaticus from H. bilis (Riley, et al. (1996) J. Clin. Microb. 34: 942-946). Briefly, a fresh fecal pellet was collected and suspended in 1.0 ml of PBS. The fecal suspension was centrifuged at 700×g for 5 minutes and 60 μl of the suspension was combined with 140 μl of PBS. Purified DNA from the fecal supernatant was obtained following the Qiagen DNeasy Tissue Kit protocol for blood (Beckwith, et al. (1997) J. Clin. Microb. 35: 1620-1623). Following PCR amplification using Helicobacter genus-specific primers, amplicons were digested separately with MboI and H-haI restriction enzymes.

Hyperimmune antisera to recombinant proteins were generated by subcutaneous injection of 20 μg of recombinant protein in 0.1 ml Freund's complete adjuvant, followed by two boosts of 10 μg of protein each in incomplete Freund's adjuvant at two week intervals. Sera were collected and tested by ELISA and antibody reactivity of antiserum was verified at a serum dilution of 1:100,000.

Native bacterial antigens: To prepare whole cell lysates, broth cultures of H. bilis, H. hepaticus, H. muridarum, B. burgdorferi, and C. jejuni were pelleted by centrifugation, washed with cold phosphate buffered saline (PBS), and then sonicated to lyse cells. Laemmli Sample buffer (Bio-Rad laboratories, Hercules, Calif.) was added to the lysates and stored at −20° C. Membrane antigen extracts of H. bilis were prepared as described (Livingston, et al. 1997. J. Clin. Microb. 35: 1236-1238). Briefly, bacteria were incubated for two days at 37° C. in Brucella broth with 5% fetal calf serum on a shaker. Bacterial cells were pelleted by centrifugation, washed with PBS and resuspended in PBS with 1% n-octyl-β-D-glucopyranoside (Sigma Diagnostics, Inc., St. Louis, Mo.) to release membrane proteins, and then centrifuged to remove insoluble protein. The supernatant was dialyzed to remove detergent.

H. bilis genomic expression library: Genomic DNA was isolated from H. bilis and 200 μg of DNA was shipped to Stratagene, La Jolla, Calif. to construct a λZAP II H. bilis genomic expression library. The λZAP II phage contains pBluescript that can be excised and cloned directly with ExAssist helper phage (Stratagene, La Jolla, Calif.). The library was screened with immune sera from H. bilis-infected mice. Immune sera were preabsorbed with E. coli/phage lysates to remove background reactivity. Immunoreactive clones were obtained by routine procedures as described (Feng, et al. (1995) Infect. Immun. 63: 3459-3466). DNA sequencing was performed at the W. M. Keck Foundation Biotechnology Resource Laboratory at Yale School of Medicine. DNA sequence was analyzed using the MacVector program (Kodak, New Haven, Conn.).

Expression and purification of recombinant proteins: Amplified genes were cloned in frame with the glutathione S-transferase (GT) gene into pMX, a pGEX-2T vector (Pharmacia, Pistacaway, N.J.) with a modified polylinker (Sears, et al. (1991) J. Immunol. 147: 1995-2001). The PCR-amplified DNA sequences of the recombinant DNA were confirmed by sequence comparison with the original inserts. Recombinant proteins were purified on glutathione columns and freed of their GT fusion partner by thrombin cleavage as described (Feng, et al. (1995) Infect. Immun. 63: 3459-3466).

Immunoblots: Four μg of membrane extract lysate or recombinant proteins were resolved in 12% SDS-polyacrylamide gels by electrophoresis and transferred to nitrocellulose membranes. For dot blots, Bio-Dot Microfiltration Apparatus (Bio-Rad, Hercules, Calif.) was used to transfer proteins to nitrocellulose membranes. A sheet of Bio-Rad 9×12 cm “Trans-Blot Transfer Medium” nitrocellulose paper was soaked for 10 minutes in TBS and then blotted with Whatman paper to dry. The Bio-Dot apparatus was assembled according to manufacturer's instructions. 100 μl of TBS was applied to each well to re-wet the membrane and then a vacuum was applied to the apparatus to remove the TBS. Protein were diluted in TBS at 10 μg/ml and 100 μl was added to each well (one well is equivalent to one dot). The TBS was allowed to pass through the nitrocellulose by gravity filtration (approximately 1.5 hours). Once all the TBS had filtered through, the unit was disassembled and the nitrocellulose were then processed as regular immunoblots.

Membranes were probed with immune serum diluted 1:100, then labeled with alkaline phosphatase-conjugated goat anti-mouse IgG secondary antibody, diluted at 1:4,500 (Sigma, St. Louis, Mo.).

Enzyme-linked immunosorbent assay (ELISA): One hundred μl of 1 μg/ml membrane extract lysate or recombinant protein in carbonate coating buffer (pH 9.6) were plated in 96 well plates as described (Feng, et al. (1998) Infect. Immun. 66: 2827-2835). Duplicate samples of each mouse serum, including uninfected normal mouse serum as a control, were diluted 1:200 for probing. Secondary antibody was alkaline phosphatase-conjugated goat anti-mouse IgG diluted at 1:5,000 (Jackson ImmunoResearch Lab. Inc., West Grove, Pa.). Optical density values were read on a Kinetic Microplate Reader (Molecular Devices, Sunnyvale, Calif.). Values were subtracted from background reactivity against normal mouse serum at OD405.

Luminex Multiplex System: Luminex beads and Luminex-100 instrument were manufactured by Luminex Corp. (Austin, Tex.). Up to a hundred distinct bead sets are available from Luminex Corp. that are identifiable by a unique fluorescent signature for each bead set detected by Luminex-100 instrument. The identity of each bead set in combination with the known protein coated onto a particular bead set provides the basis for multiplexing capabilities of the Luminex detection system.

Coupling Proteins to Luminex beads: Luminex beads were coated with various proteins by chemical cross-linking according to manufacturer's instructions. Bead stock was resuspended by vortexing and sonication (15 to 30 sec.). An aliquot of 2×10⁶ beads was removed and centrifuged at 12,000×g for 2 min. Beads were resuspended in 80 μl of activation buffer (100 mM monobasic sodium phosphate; pH 6.3) by vortexing and sonication (15 to 30 sec.).

Activation of beads for protein coupling: To prepare the beads for cross-linking to proteins, 10 μl of 50 mg/ml Sulfo-NHS (N-hydroxysulfosuccinamide; Pierce, Rockford, Ill.) was added and beads were mixed by vortexing. Then 10 μl of 50 mg/ml EDC (1-ethyl-3-[3-dimethylaminopropyl] carbodiimide; Pierce, Rockford, Ill.) was added and beads were mixed again by vortexing. The bead mixture was incubated shaking on a rocker at room temperature (RT) for 20 min and centrifuged at 12,000×g for 2 min. Beads were washed twice with 1 ml of 50 mM MES (pH 6.0) buffer. To coat with proteins, pelleted beads were resuspended in the relevant protein solution diluted in 50 mM MES (pH 6.0) buffer. Protein preparations were diluted to the final concentration as follows:

1 and 10 μg/ml of P167C

1 and 10 μg/ml of membrane extract from H. bilis

1 and 10 μg/ml of membrane extract from H. hepaticus

Beads were also coated with a control antibody (biotin conjugated Goat IgG, 100 μg/ml) and a control protein (bovine serum albumin (BSA, 100 μg/ml) diluted in 50 mM MES (pH 6.0) buffer. Mixture of activated beads and proteins was incubated shaking on a rocker for 2 hr at RT for coupling. After coating with proteins, beads were washed twice with wash buffer (0.1% Tween-20 in phosphate buffered saline (PBS), pH 7.4) and resuspended in 1 ml of blocking buffer (1% BSA; 0.1% Tween-20 in PBS, pH 7.4; 0.05% sodium azide). Blocking was performed by shaking on a rocker at room temperature for 30 min. After blocking, beads were washed twice in 1 ml blocking buffer. Finally, coupled beads were resuspended in 1 ml blocking buffer and stored at 4° C. for up to a week. For long-term storage beads were kept frozen at −70° C. for several months.

Multiplex detection of antibodies in mouse serum by the use of Luminex System: Immunoreactions were set up in 96 well, filter bottomed plates designed for high throughput separations (1.2 μm MultiScreen, Millipore Corporation, Bedford, Mass.). Typically, up to 1000 beads for each individual bead set, coated with a known protein, were added per well. For example, for a six-plex assay, 1000 beads from each set coated with a known antibody were mixed to provide a total of 6,000 beads. This six-plex bead mixture was added per well in a total of blocking buffer. To this, 50 μl of mouse serum, diluted 1:100 in 5% blotto, was added. The mixture of beads and diluted serum was then incubated on a shaker for 1 hr at RT.

After incubation, liquid was drained from the bottom of plate, under vacuum, on a suction apparatus (Millipore Corporation) designed to fit these plates. The beads were washed three times by adding 100 μl of wash buffer per well and draining out under vacuum successively. For detection of mouse IgG, biotinylated goat anti-mouse IgG was used (Vector laboratories, Burlingame, Calif.). This secondary antibody was diluted 1:1000 in wash buffer and 100 μl was added per well. Beads were mixed as before and incubated at RT for 30 min. Following incubation with the secondary antibody, beads were washed three times as before. To detect biotinylated IgG, 100 μl of streptavidin conjugated to R-phycoerythrin (CalTag, Burlingame, Calif.) was added at a dilution of 1:500 in wash buffer. The contents of each well were mixed and incubated at RT for 15 min. Beads were washed three times with wash buffer. Washed beads were finally resuspended in 100 μl of wash buffer per well and analyzed in the Luminex-100 instrument.

Luminex-100 Operation and Data Analysis: Luminex-100 instrument was used at default settings, set by the manufacturer for routine use, as directed by the User's Manual. The instrument came with a complete software package for the operation of instrument called Luminex Data Collection Software. The software package allowed routine operation, data acquisition, and data analysis. In this invention, Version 1.7 of the software was used according to instructions in the User's Manual supplied by the manufacturer. After initial analysis using Luminex software, data was plotted by the use of Microsoft Excel software.

### Example 2

### Identification of Immunodominant H. bilis Antigens

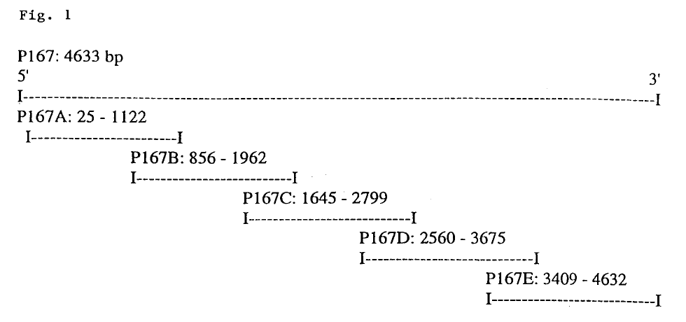

Immune sera from mice that were experimentally infected with H. bilis at six months after gavage inoculation reacted to H. bilis membrane antigen extracts by ELISA at a dilution of 1:800. Immunoblots with membrane antigen extracts revealed reactivity of six month immune serum against 5 major bands with approximate molecular weights of 15, 30, 40, 50, 60 kDa, and 18 minor bands ranging from 10 to 100 kDa (FIG. 1.) These results confirmed that serum from experimentally infected mice were reactive against a number of H. bilis antigens.

The H. bilis genomic DNA expression library was then probed with six month immune serum. Seventeen immunoreactive clones were obtained and sequenced. One clone encoded a 4633 base pair (bp) open reading frame (P167), and the gene product's predicted molecular weight was 167 kDa. The open reading frame had typical bacterial −10 (TTGTAA) and −35 (TATAAA) potential promoters and a ribosomal binding site (AAAAGAG) on the 5′ flanking region, a stop codon, and a translation terminator: a hairpin structure in the 3′ flanking region. Another clone contained a gene (P158) encoding a product with a predicted molecular weight of 158 kDa. This gene shared an identical fragment of 2877 bp from 1490-4366 bp corresponding to P167 from 1756-4633 bp. P158 is 4366 bp long, and is 267 bp shorter from its 5′ end than P167. The first 1100 bp of P158 share very low, if any, homology with P167. The midsection of P158 (nucleotides 1101-1489) shares 52% identity the P167 counterpart, and the 3′ section of P158 is 100% identical to that of P167. Among the seventeen different immunoreactive clones, thirteen were determined to contain either P167 or P158. Three clones contained the complete sequence of P167; six clones contained partial sequences of P167; and three clones contained the complete sequence of P158. The remaining four clones reacted with antiserum to P167C. Therefore, they were not sequenced. These results suggest that one or both of these proteins (P167 and P158) are dominant immunoreactive antigens during H. bilis infection. Sequences of P167 and P158 have been submitted to the GenBank Nucleotide Sequence Databases. The Genbank Accession numbers for P167 and P158 are AF288477 and AF349728, respectively.

In summary, a large, immunoreactive protein, P167 was identified. P167 elicits an antibody response in mice infected with H. bilis. Comparison of membrane extracts with whole cell lysates as antigens on immunoblots revealed that antiserum to P167C reacted weakly with cell lysates, but not with the membrane antigen preparation. This suggested that P167 may not be associated with the membrane, and may be a secretory product that stimulates host immunity. Alternatively, P167 may be preferentially expressed in vivo, but not in vitro, or may be a secretory product. Thus, despite the logic of focusing on membrane proteins as potential antigens for serodiagnosis, other antigens may be superior in terms of antigenicity, species specificity, and possibly sensitivity.

### Example 3

### Analysis of P167 Fragments

Five overlapping peptides of P167 (encoded by SEQ ID NO: 1) were created (FIG. 2): P167A, P167B, P167C, P167D, and P167E. P167A was created from nucleotides 25 to 1122 of SEQ ID NO: 1 and had a predicted molecular weight of 39 kDa. P167B was created from nucleotides 856 to 1962 of SEQ ID NO: 1 and had a predicted molecular weight of 40 kDa. P167C was created from nucleotides 1645 to 2799 of SEQ ID NO:1 and had a predicted molecular weight of 41 kDa. P167D was created from nucleotides 2560 to 3675 of SEQ ID NO: 1 and had a predicted molecular weight of 41 kDa. P167E was created from nucleotides 3409 to 4632 of SEQ ID NO: 1 and had a predicted molecular weight of 45 kDa. Template DNA from the original reactive clone was used to amplify the gene fragments. 11811 Immune serum from experimentally H. bilis-infected mice reacted on immunoblots against two of the five peptide fragments, P167C and P167D (FIG. 1, lanes 2 and 4). Immune serum reacted against recombinant P167C and P167D at dilutions of 1:1600 and 1:6400, respectively, by ELISA. Both reacted at higher titer than the membrane extract (1:800) when tested simultaneously.

P167C antiserum, generated by hyperimmunization of mice, reacted strongly with recombinant P167C and only weakly with H. bilis whole cell lysate-immunoblots against a protein band with a molecular weight of approximately 120 kDa, which apparently represented native P167. A protein band of this size was not seen on immunoblots prepared from H. bilis membrane extract. These findings suggested that P167 may be preferentially expressed in vivo, or is a secretory product, or both. To test for specificity of P167 for H. bilis, we next examined immunoblot reactivity of P167C antiserum against whole cell lysates of H. hepaticus and H. muridarum, as well as two unrelated spirochetal species, B. burgdorferi and Campylobacter jejuni. The Campylobacter genus is closely related to Helicobacter and was at one time included in the Helicobacter genus (Goodwin, et al. (1989) Int. J. Syst. Bacteriol. 39: 397-405; Jerris, R. C. Manual of Clinical Microbiology, eds: Murray, et al., (1995) American Society for Microbiology: Washington, D.C. p. 492-498). P167C antiserum did not react with bands of similar size to P167 from H. hepaticus, H. muridarum, B. burgdorferi or C. jejuni whole cell lysates. P167C and P167D peptides probed with immune serum from experimentally infected mice with H. hepaticus, this serum had no cross reactivity with P167C or P167D (FIG. 1, lane 3 and 5). These data suggest that at least the antigenic epitope(s) of P167 is (are) H. bilis-specific.

PCR was performed with all five sets of P167 fragment primers, using H. hepaticus and H. muridarum genomic DNA as templates. All five fragments were amplified from H. muridarum genomic DNA and the amplicons were similar in size to those of H. bilis (FIG. 3). In contrast, P167A, P167B, P167C, and P167D primers amplified DNA from H. hepaticus, but P167E primers did not. The amplification products were of equivalent size to their corresponding H. bilis homologues. No product was amplified with any of the five primer sets from C. jejuni or B. burgdorferi genomic DNA target. The density of amplicons from H. hepaticus and H. muridarum was much less than that of H. bilis, suggesting lower homology of P167 counterparts in H. hepaticus and H. muridarum.

### Example 4

### Determination of Whether Naturally Infected Mice had Antibodies Reactive to Recombinant P167

Sera were collected from nine mice that had PCR-verified natural infections with H. bilis, and from four mice that had PCR-verified natural infections with H. hepaticus. The duration of infection in these mice was unknown. These sera were probed on dot immunoblots against H. bilis membrane extract, P167C, and P167D (FIG. 4).

Sera from four of the nine H. bilis-infected mice reacted with all three antigens, and sera from one of the nine H. bilis infected mice reacted with only the membrane extract. Sera from one H. bilis infected mouse reacted to P167D only, and sera from another H. bilis infected mouse had antibodies that reacted to both P167C and P167D. Two mice infected with H. bilis developed antibodies neither to H. bilis membrane extract nor to P167C or P167D. Sera from the four H. hepaticus-infected mice did not react with P167C or P167D, but sera from one of the four H. hepaticus-infected mice cross-reacted with H. bilis membrane extract.

In summary, of the seven H. bilis-infected mice that reacted to any one of the antigens six of the mice reacted to P167D, five of the mice reacted to P167C, and five of the mice reacted to membrane extract. These results show the usefulness of these recombinant antigens to serve as diagnostic reagents.

Comparison of serum reactivity titers to membrane extract with recombinant P167C and P167D peptides revealed higher titers to the recombinant peptides, with the added advantage of species specificity. Further studies are needed to determine if P167C/P167D can detect early antibody responses during infection. Testing a limited number of serum samples from naturally infected mice suggested that membrane antigen extracts were less sensitive (and less specific) for detecting antibody reactivity to H. bilis.

P167C hyperimmune serum did not recognize a protein of similar size to P167 from H. hepaticus, or H. muridarum whole cell lysates, and H. hepaticus immune serum did not react with P167C or P167D recombinant proteins by immunoblotting. The fact that all five fragments of P167 could be amplified from H. muridarum genomic DNA, and four out of five fragments of P167 (P167A, P167B, P167C, and P167D) could be amplified from H. hepaticus, indicates that H. bilis, H. hepaticus, and H. muridarum all share homologues of this gene that do not share antigenic epitopes. Furthermore, at least three clones that contained an operon that encoded a predicted 158 kDa gene product that shared regions of homology with P167. This might indicate that recombination may occur at a variable mid section of the gene to create new genes. What drives the recombination is not clear, but may suggest these proteins have important functions in the biology of this bacterium.

### Example 5

### Identification of P17Hb48, a Helicobacter bilis Antigen

P17Hb48 was identified from a Helicobacter bilis genomic expression library as described in Example 1 above. The Genbank Accession No. for P17Hb48 is AF444005.

To verify that amplification of P17Hb4 is specific for H. bilis, DNA was purified from mouse feces and used as templates, and P17Hb48 primers (5′primer: ATGGAACAGATAAAGATTTTAAAGCAACTTCAG (SEQ ID NO: 16) and 3′primer: CTATGCAAGTTGTGCGTTAAGCAT (SEQ ID NO: 17)) were used to amplify P17hb48 genes. DNA was denatured at 94° C. for 1 minute, annealed at 55° C. for 1 minute, and extended at 72° C. for 1 minute. This process was repeated for 30 cycles. Amplicons were analyzed by gel electrophoresis. P17Hb48 DNA was only amplified from H. bilis infected mice, and not from H. hepaticus infected mice.

### Example 6

### Identification of P25Hh4, a Helicobacter hepaticus Antigen

Helicobacter hepaticus genomic expression library constructed as described for the H. bilis library described in Example 1 above. P25Hh4 was identified from by screening the library with immune sera from H. hepaticus infected mice as described in Example 1 above. The Genbank Accession No. for P25Hh4 is AF444004.

To verify that amplification of P25Hh4 is specific for H. hepaticus, DNA was purified from mouse feces and used as templates, and P25Hh4 primers (5′primer: ATGGGTAAGAAAATAGCAAAAAGATTGCAA (SEQ ID NO: 13) and 3′primer: CTATTTCATATCCATAAGCTCTTGAGAATC (SEQ ID NO:14)) were used to amplify P25Hh4 genes. Briefly, DNA was denatured at 94° C. for 1 minute, annealed at 55° C. for 1 minute, and extended at 72° C. for 1 minute. This process was repeated for 30 cycles. Amplicons were analyzed by gel electrophoresis. P25Hh4 DNA was only amplified from H. hepaticus infected mice, and not from H. bilis infected mice.

### Example 6

### Immunoassay Detection of Helicobacter Antigens in the Sera of H. bilis and H. Hepaticus Infected Mice

Immunoassays (e.g., ELISAs and Luminex assays) using P167C, P167D, H. bilis membrane extracts, or H. hepaticus membrane extracts and sera from mice infected with either H. bilis or H. hepaticus, were performed as described in Example 1 above. Infection with H. bilis or H. hepaticus was confirmed using PCR as described in Examples 5 and 6 above. Table 1 shows the results of ELISAs and Table 2 shows the results of Luminex assays.

TABLE 1

| | ELISA (positive/total) | | | |
|---|---|---|---|---|
| Antigens | P167C | P167D | H. bilis me* | H. hepaticus me* |
| Sera from H. bilis infected | 18/22 | 20/22 | 19/22 | 6/22 |
| mice | | | | |
| Sera from H. hepaticus | 0/9 | 0/9 | 3/9 | 9/9 |
| infected mice | | | | |

*me: membrane extracts.

TABLE 2

| | Luminex (positive/total) | | |
|---|---|---|---|
| Antigens | P167C | H. bilis me | H. hepaticus me |
| Sera from H. bilis infected | 17/20 | 10/20 | 6/20 |
| mice | | | |
| Sera from H. hepaticus | 0/20 | 0/20 | 2/20 |
| infected mice | | | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated polynucleotide that hybridizes under stringent conditions to a sequence selected from the group consisting of: SEQ ID NO:1, nucleotides 25-1122 of SEQ ID NO:1, nucleotides 856-1962 of SEQ ID NO:1, nucleotides 1645-2799 of SEQ ID NO: 1, nucleotides 2560-3675 of SEQ ID NO: 1, nucleotides 3409-4632 of SEQ ID NO: 1, SEQ ID NO:12, SEQ ID NO:15, or a complement thereof.

2. An isolated polynucleotide that hybridizes under stringent conditions to SEQ ID NO: 1 or a complement thereof.

3. An isolated polynucleotide that hybridizes under stringent conditions to nucleotides 25-1122 of SEQ ID NO: 1 or a complement thereof.

4. An isolated polynucleotide that hybridizes under stringent conditions to nucleotides 856-1962 of SEQ ID NO:1 or a complement thereof.

5. An isolated polynucleotide that hybridizes under stringent conditions to nucleotides 1645-2799 of SEQ ID NO:1 or a complement thereof.

6. An isolated polynucleotide that hybridizes under stringent conditions to nucleotides 2560-3675 of SEQ ID NO: 1 or a complement thereof.

7. An isolated polynucleotide that hybridizes under stringent conditions to nucleotides 3409-4632 of SEQ ID NO: 1 or a complement thereof.

8. An isolated polynucleotide that hybridizes under stringent conditions to SEQ ID NO:12 or a complement thereof.

9. An isolated polynucleotide that hybridizes under stringent conditions to SEQ ID NO: 15 or a complement thereof.

10. An expression vector comprising a polynucleotide of claim 1 operably linked to an expression control sequence.

11. A host cell comprising an expression vector according to claim 10.

12. The host cell of claim 11, wherein the cell is E. coli.

13. An isolated polypeptide comprising an amino acid sequence encoded by a polynucleotide of claim 1.

14. A method for detecting Helicobacter infection in a rodent comprising the steps of:
(a) contacting a polypeptide encoded by SEQ ID NO: 1, SEQ ID NO: 12, SEQ ID NO: 15 or an immunogenic fragment of said polypeptide with a biological sample, thereby forming a complex between the polypeptide and an antibody in the sample; and
(b) detecting the presence of the complex, thereby detecting the presence of Helicobacter infection.

15. The method of claim 14, wherein the polypeptide is encoded by nucleotides 1645-2799 of SEQ ID NO:1.

16. The method of claim 14, wherein the polypeptide is encoded by nucleotides 2560-3675 of SEQ ID NO: 1.

17. The method of claim 14, wherein the Helicobacter is H. bilis.

18. The method of claim 14, wherein the Helicobacter is H. hepaticus.

19. The method of claim 14, further comprising the step of contacting the complex with a rodent Ig-specific antibody.

20. The method of claim 19, wherein the rodent Ig-specific antibody is labeled with a detectable label.

21. A kit for detecting Helicobacter infection comprising a polypeptide encoded by SEQ ID NO:1, SEQ ID NO:12, SEQ ID NO:15, or an immunogenic fragment of said polypeptide.

22. The kit of claim 21, further comprising a rodent Ig-specific antibody.

23. The kit of claim 22, wherein the rodent Ig-specific antibody is labeled with a detectable label.

24. A method for detecting Helicobacter infection, said method comprising the steps of:
(a) amplifying a target nucleic acid sequence with a first primer and a second primer specific for Helicobacter nucleotide sequence; and
(b) detecting said amplified product.

25. The method of claim 24, wherein the target nucleic acid is from H. bilis.

26. The method of claim 25, wherein the first primer hybridizes to the sequence set forth in SEQ ID NO: 16 and the second primer hybridizes to the sequence set forth in SEQ ID NO:17.

27. The method of claim 25, wherein the first primer comprises the sequence set forth in SEQ ID NO: 16 and the second primer comprises the sequence set forth in SEQ ID NO:17.

28. The method of claim 25, wherein the first primer and the second primer are independently selected from the group consisting of SEQ ID NOS:2, 3, 4, 5, 6, 7, 8, 9, 10, 11, 12, 16, and 17.

29. The method of claim 24, wherein the target nucleic acid is from H. hepaticus.

30. The method of claim 29, wherein the first primer hybridizes to the sequence set forth in SEQ ID NO: 13 and the second primer hybridizes to the sequence set forth in SEQ ID NO:14.

31. The method of claim 29, wherein the first primer comprises the sequence set forth in SEQ ID NO: 13 and the second primer comprises the sequence set forth in SEQ ID NO:14.
